(19) United States
(12) Patent Application Publication
(10) Pub. No.: US 2005/0049498 A1
(43) Pub. Date: Mar. 3, 2005

(54) Method of ultrasound non-contact early detection of respiratory diseases in fowls and mammals

(75) Inventors: Robert H. Roche, Westminster, MD (US); Vadym Buyalsky, Reisterstown, MD (US)

Correspondence Address: Robert H. Roche;CTRL Systems, Inc., Suite H, 1004 Littlestown Pike, Westminster, MD, 21157-3026, US

(73) Assignee: CTRL Systems, Inc., Westminster, MD (US)

(21) Appl. No.: 10/917,012

(22) Filed: Aug. 12, 2004

Related U.S. Application Data
(60) Provisional application No. 60/494,830, filed on Aug. 13, 2003.

(51) Int. Cl. A61B 8/00

(52) U.S. Cl. 600/437

(57) ABSTRACT

In the case of disease in the respiratory organs of creatures, which especially inhabit in terms of overcrowding, the danger of spreading a disease throughout the entire breeding farm is perfectly evident. The use of traditional methods, such as the stethoscope, for examining the respiratory organs of each creature, in the mode of their direct contact listening, is impossible in most due to the specific spatial arrangement of creatures in breeding farms and/or infectious danger to a veterinary. Moreover, the acoustic signs of the said illness at its early stage cannot be heard audibly with use of traditional methods. The present invention provides a novel method of ultrasound non-contact early detection of respiratory diseases in fowl and mammals, and other breathing creatures of animate nature, wherein innovative technology is applied for revealing the suspected minimal unit group with sick creature(s); wherein the said technology includes at least the following interrelated techniques: I-Stationary 3-D Surveying Technique that enables to identify vectored direction(s) to suspected sections in total creatures' inhabiting area by electronically scanning the said total area with 3-D array of ultrasonic transducers. II-Portable 2-D Inspection Technique that permits to verify disposition of perfect suspected sectional area by locating around each suspected inhabiting section with portable and hand-held ultrasonic detecting devices. III-Local 1-D Pinpointing Technique that provides for pinpointing a distinct suspected unit group, being inside an infected inhabiting sector, by a hand-held ultrasound pick-up device, equipped with extending acoustic probes for veterinary safety. Portable and Local Techniques optionally provides for use of wireless processing with Data Processing System. Spatio-Temporal compliance in applying the said techniques is being considered by operational algorithm of Data Processing System.

### REFERENCES CITED

| | | |
|---|---|---|
| 5,930,201 | July 1999 | Cray |
| 6,189,384 | February 2001 | Oiety et al. |
| 6,565,510 | May 2003 | Haider |
| 6,567,682 | May 2003 | Osterweil et al. |
| 6,582,367 | June 2003 | Robinson et al. |
| 6,597,934 | July 2003 | de Jong et al. |

### Other References

“Highly Pathogenic Avian Influenza”—USDA, Animal and Plant Health Inspection Service, Veterinary Service, February 2002.

“NC228: Avian Respiratory Diseases: Pathogenesis, Surveillance, Diagnosis and Control. Available from the web: http://www.lgu.umd.edu/proiect/home.cfm?trackID=1514. Dated: Jul. 29, 2004.

### FIELD OF THE INVENTION

The invention refers to detection of ultrasonic radiation that is being emitted by creatures of animate nature, and more particularly to early and non-contact, infectiously safe detection of typical ultrasound signs of respiratory diseases in fowls and mammals, which are being bred at farms, cared inside open-air cages and enclosures, or watched in wild nature.

### BACKGROUND OF THE INVENTION

There are specific conditions involving the breeding of fowls and mammals where a random illness of a creature may raise an epidemic and therefore devastate the efficiency and profitability of a breeding farm. In the case of disease of the respiratory organs of these creatures, there exists the evident danger of spreading a disease throughout the entire breeding farm. It is necessary therefore to find a sick or diseased creature as early as possible. However, the use of traditional stethoscopes for examining each creature in the mode of contact listening to their respiratory organs is impossible due to the design of such farms and arrangement of creatures in current breeding processes. Therefore, the acquisition in proper time and in non-contact manner of all the typical acoustic signs of respiratory diseases should be of great anti-epidemic and commercial importance. This problem may be solved on the basis of the phenomena that at the early stage of said illness its acoustic signs cannot be heard audibly, but can be heard, detected and recorded in the ultrasound range either automatically by a stationary 3-D array of ultrasonic transducers or by an examiner, equipped with a hand-held ultrasonic device. Since ultrasound waves propagate in air from sickly breathing creature to the remote examiner's position, this phenomenon enables a veterinary to avoid the present diagnosis difficulties and infectious threat, caused by the mentioned above epidemic hazards. The small-scale farm breeding of fowls and mammals assumes the absence of their chaotic movement inside farm building. Such spatially restricted arrangement of creatures enables the examiner to review and watch them systematically, in particular diagnosing their bodies by direct contact. The regular industrial farm breeding of large flocks of fowls and some kinds of mammals also assumes prevention of their movement inside farm building in chaotic manner, but at the same time the arrangement of these creatures in a tight adjacency hinders the examiners from accessing each creature's body separately either for observing this body visually or for diagnosing their respiratory organs by regular direct hearing with stethoscope for finding any signs of illness. Besides, the constant audible noise inside the farm building prevents the ability to hear any signs of respiratory disease in acoustic range available for human hearing. In such conditions, it is reasonable to use the advantages of ultrasound, which is created through the breathing of creatures with respiratory disease. These advantages of ultrasound include, in particular, the ability to propagate through air for enough long distances for detection and to not be influenced by surrounding audible “white noise”. So, in accordance with the present invention, ultrasound non-contact diagnosing is the new method of early detection of respiratory diseases in fowls and mammals. The nearest ancestor of the said method is the method that has been discovered by U.S. Pat. No. 6,189,384 B1, where ultrasonic monitoring is used for a progressive surveying of machines' degradation development until the permissible wear rate, and where this ultrasound monitoring is based on the predicted spatio-temporal routing, which is being planned by central processing system in dependence on the results of previously and later acquired data comparison. Such a methodology couldn't be applied in terms of unpredictable spatio-temporal parameters of the mentioned above respiratory diseases occurrence and spread. The operating regime of entire system and its functional components must be submitted to the terms of urgent detection of area and sequent pinpointing of place where random appearance of alarm ultrasound signs of respiratory illness have occurred.

The suggested by the present invention method of ultrasound diagnosing of respiratory diseases in fowls and mammals, and other breathing creatures of animate nature has been made free of the mentioned above disadvantages, since there are being purposefully used as innovative techniques of non-contact early ultrasound detection of respiratory diseases of said creatures, as novel interrelation among those 3-D surveying, 2-D inspection and 1-D pinpointing techniques. The successful putting this method into practice should help to avoid annual loss of scores of millions dollars in poultry husbandry of the United States of America.

### SUMMARY OF THE INVENTION

The present invention provides for a novel method of ultrasound non-contact early detection of respiratory disease in fowls and mammals, and other creatures of animate nature wherein the operating regime of entire system and of each component of this system must be devoted to:
I-scanning inside 3-D room of the total inhabiting area with the aim of vectored location of a suspected section with a sick creature;
II-verifying by 2-D motion of portable detectors the suspected sectional position where the ultrasound signs of illness are being emitted from; and
III-pinpointing by 2-D/1-D motion of portable detectors a suspected unit group with sick creatures.

It is the principle object of the present invention to provide a relevant interrelation of ultrasound techniques for non-contact detecting of non-audible signs of respiratory disease for creating a method of distinguishing sick creatures from healthy creatures, e.g. in a large-size flock at the breeding farm.

Another object of the invention is to provide a technique for sampling the typical acoustic signs of respiratory illness of fowls, mammals and other creatures in the form of ultrasound waves that propagate in air over numerous flocks and that pertain to the early beginning stage of an illness.

A further object of the invention is to provide a technique of sequential procedures that enable the examiner to distinguish the sick creature, including:
Initial vectored determination of the area from which the ultrasound signal is being radiated;
Location of the specific zone that reveals the more intensive ultrasound signal from the flock;
Pinpointing inside the suspected zone the unit group of creatures, which the most intensive typical ultrasound signals come from;
Finally, accessing to and pinpointing of the sick creature inside the suspected unit group of fowls, mammals, or other beings of animate nature.

Still another object of the invention is to provide a schedule of operating interrelation among the procedures of acquisition of running ultrasound signals for comparison with said preliminary sampled ultrasound signals, where such comparison should result in moving away the infected unit group or even a few sick creatures from an entire flock.

### BRIEF DESCRIPTION OF THE FIGURES

Predominant embodiment of the present invention will be described herein with reference to the figures by way of graphical illustration, in which fundamental arrangement of the suggested innovative method is represented, and in which explanations of said arrangement are given.

FIG. 1 represents the novel spatio-temporal interrelation of non-contact ultrasonic detecting techniques where the priority of locating and pinpointing a respiratory sick unit group or even a few creatures belongs to portable hand-held measures, which are being dispatched in a time interleaved manner by an automatic 3-D scanning measure thru a typical Data Processing System, adapted to the random distribution of accidental ultrasound signs of respiratory diseases in fowls, mammals and other breathing creatures.

FIG. 2 displays the graphic representation of spatially vectorial aiming of beam patterns of portable ultrasound devices during implementation of Portable 2-D Inspection Technique, and the extending set of acoustic probes at the time of fulfilling the Local 1-D Pinpointing Technique.

### DETAILED DESCRIPTION OF PREFRRED EMBODIMENTS

The preferable embodiments of the present invention are the interrelated techniques of the novel method of ultrasound non-contact diagnosing of respiratory organs of fowls, mammals and other creatures with the aim of early detection of diseases thereof. The following description is expected to deliver the apt explanation of embodiments, advantages and benefits of the method claimed herein.

The ultrasonic signs for the mentioned above purpose may be defined as the following high frequency acoustic evidences of the early stage of respiratory illness:
Hard dry breathing with high frequency vibration of epithelium in sick respiratory organs;
Light sneezing with cavitations in saliva and coughing that produce ultrasound;
Wheezing in lungs as a result of phlegm loosening with breathing; and
Turbulence in water suction from creatures drinking to quench excessive thirst.

According to the method of the present invention, the sampling of these acoustic evidences is being accomplished in the form of combined ultrasonic images that:
Contain the patterns of ultrasonic vibration of each pertaining sign and the pattern of background noise, which describes either common or local sound condition at the farm or at another place of creatures' inhabiting;
Those images are being acquired separately but used either separately or in proper combination according to the procedure of diagnosing.

In compliance with the method of the present invention, the technology of distinguishing a unit group (or even a few) of sick creatures is being carried out with interacting techniques that provide for narrowing of search zone in consecution: direction to suspected section of an entire creatures' inhabiting area/—direction to suspected sector of a suspected section/—place of suspected unit group.

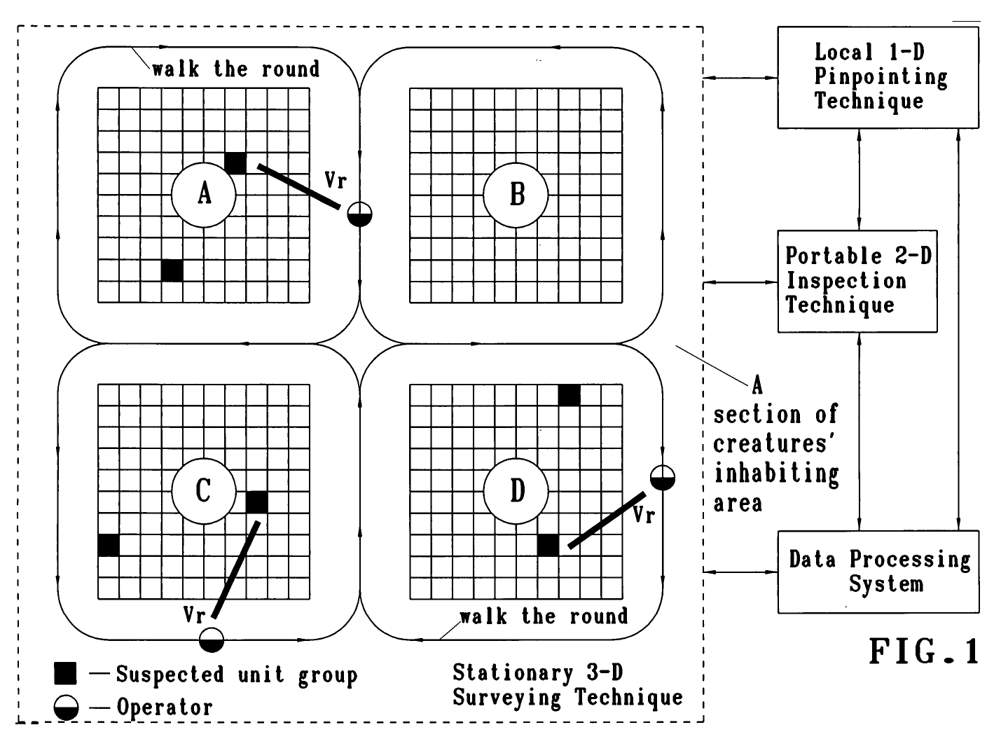

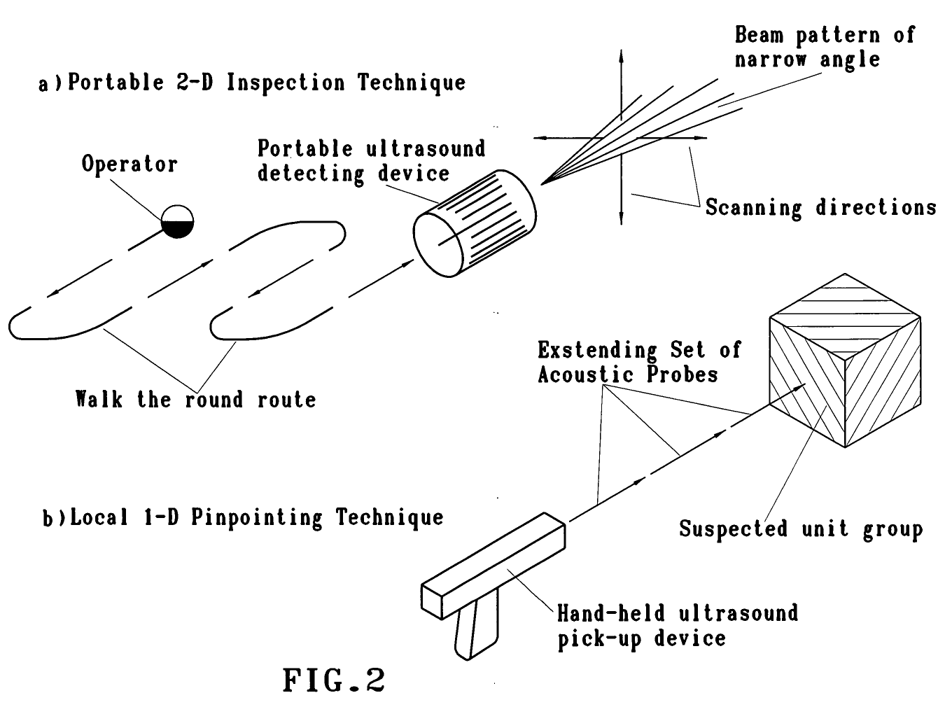

FIG. 1 shows an example of a section of an entire creatures' inhabiting area, which consists of some sectors A, B, C, and D, and which is being in continuous interaction with Data Processing System (DPS). Allocation of surveyed creatures in sections, sectors, and unit groups prevents their chaotic motion and therefore enables the systematized ultrasound search for at least a unit group of creatures that emit ultrasonic signs of a respiratory disease. Stationary 3-D Surveying Technique is being applied for the constant search of vectorial direction toward a suspected section or sector. The preferable embodiment of the said technique should consist of a stationary array of ultrasound transducers, whose spatial arrangement provides for filling all the surveyed space (area, section, sector) with beam patterns of those transducers with space factor not less than 1.2, and whose specification figures (at least: directivity, sensitivity, selectivity, remote ability, and S/N ratio) correspond to the operational conditions of acquiring ultrasound signs of respiratory diseases. The vectorial direction toward a suspected zone, where the sampled ultrasound emission comes from, is being defined by DPS in both ways: by indexing this zone, e.g. sector A, B, C or D in a proper numbered section, see FIG. 1, and by stating a direction from nearest operator to the suspected zone (preferably suspected sector), hence the said operator and sector happened to be adjacent at the moment of the said signal appearance. The priority is to be given to such a direction that leads to the central part of a suspected sector. The frequency of inquiry of each ultrasound transducer in 3-D electronic scan is being rated at least as a product of the average frequency of normal creature's breathing and total number of creatures in an inhabiting area. FIG. 1 illustrates the constant double-way interaction among all the techniques with the aim of fast and trustworthy distinguishing a unit group of sick creatures (or even a few of creatures). Certainly, at least three operators per section are being on their scheduled walk the round routes all the time. They continuously fulfill Portable 2-D Inspection Technique that includes short-distance 2-D scanning side to side in passages between sectors and up-and-down the multi-storey racks of sectors with portable ultrasound detecting device, see FIG. 2. Those devices should possess the beam pattern angle preferably not more than 25° for faultless location of a suspected unit group. Such a value of the beam pattern angle may be achieved by use of ultrasound concentrators (parabolic, conical, etc.). Once DPS fixes that Stationary 3-D Surveying Technique has found a direction toward the suspected sector, it should choose an operator, which was on his walk the round route and happened to be adjacent to said found suspected sector (or even found suspected part of this sector). In the result of having got an order from DPS the said operator changes his routine walk the round route for vectored direction Vr and applies Portable 2-D Inspection Technique for short-distance location around the suspected sector with the aim of detecting the most suspected place therein, see FIG. 1. While interacting with data-base of DPS, the operator has proved the presence of ultrasound signs of respiratory disease inside a suspected sector, he should deploy Local 1-D Pinpointing Technique. This 1-D technique is being accomplished with use of a hand-held ultrasound pick-up device, which must be equipped with extending set of acoustic probes, see FIG. 2, protected from a competitive ultrasound by any known measures. If the suspected unit group is trustworthily pinpointed by applying Local 1-D Pinpointing Technique, the proper alarm activating signal is being released and the involved operator stays thereat till sick creatures have been withdrawn by a special staff. Should the number of vectored by 3-D technique the suspected directions will exceed the number of operators, all the competing ultrasound sources are to be stopped for repeated verification of suspected directions and for making a reasonable decision. During application of Local 1-D Pinpointing Technique in one of the sectors another 3-D and 2-D techniques continue to function in accordance with scheduled routine.

In accordance with the method of the present invention, the processing of signals, that were acquired during diagnosing, and signals, retrieved from a preliminary formed data base of ultrasound images, is being fulfilled basically as follows:
Choosing the foreground form of acquired ultrasound signals for comparison with said preliminary sampled ultrasound images;
Comparing acquired and retrieved signals simultaneously and using the result of said comparison for correction of vector of search, either for correction of reading combinations at array of ultrasound detectors or for correction of scanning space trajectory of at least one portable ultrasonic receiver. It is evidently expected that the method according to the present invention will be used successfully for non-contact inspection in order to detect and identify a creature with sick respiratory organs at farms for breeding fowls, mammals and other creatures of animate nature. Additionally, the present invention could also be implemented (in full or in part) at zoo and national park settings for non- contact and safety diagnosis of animals, which are arranged in flocks or distributed over a terrain. The said evidence is based on the cost-benefit and trustworthy features of the method of the present invention, because:
It is a suitable replacement for the labor-intensive and hazardous contact diagnosis of suspected individual creatures inside large-size flocks; and
The present non-contact ultrasonic technology invention is a suitable replacement for subjective audible diagnosis of respiratory organs of creatures of animate nature.

The present invention is not to be confined to the precise details herein described, nevertheless changes and modifications may be made so far as such changes and modifications indicate no significant deviations from the sense and art of the claims.

1. Method of ultrasound non-contact early detection of respiratory diseases in fowls and mammals, and other breathing creatures, which provides for the diagnosis of their respiratory organs at an infectious safe distance by non-contact inspection and recording acquired ultrasound signals, and analyzing said signals in comparison with ultrasound images that pertain to the early beginning stages of respiratory diseases.

2. Method as defined in claim 1 wherein ultrasonic signs and evidences of early diseases of respiratory organs (hard dry breath, light sneezing, coughing, wheezing, sipping to quench excessive thirst, etc.), are being preliminarily identified and sampled from certainly sick creatures in ultrasound images, recorded in real conditions of ultrasound inspection in creatures' inhabiting areas and at distances that enable the infectious safe diagnosing, where said sampling process is being done while preventing any chaotic and mixing movement inside a total crowd of creatures by any known method.

3. Method as defined in claim 1 wherein the vectorial location of ultrasonic signs of a respiratory disease is being fulfilled by scanning electronically throughout the total creatures' inhabiting area with applying Stationary 3-D Surveying Technique that utilizes 3-D array of ultrasound transducers, where redundant spatial arrangement of said transducers in said inhabiting area provides that this area is being surveyed with the space factor not less than 1.2 and where the frequency of inquiry of each ultrasound transducer in electronic scan is being rated regarding at least the average frequency of normal creature's breathing and total number of creatures in an entire inhabiting area.

4. Method as defined in claim 1 wherein the ultrasound search throughout sections of an inhabiting area for the perfect position of sector with sick creatures is being carried out by scanning with Portable 2-D Inspection Technique towards the previously vectored directions that consists in locating around each suspected inhabiting sector with portable ultrasound detecting devices, which possess the properly rated directivity, sensitivity and remote ability and which interact with 3-D array of ultrasonic transducers by means of Data Processing System.

5. Method as defined in claim 1 wherein the final identification of suspected unit group in the sector(s), determined as an infected, is being accomplished by pinpointing of local unit group position with Local 1-D Pinpointing Technique, where there are being used hand-held ultrasound pick-up devices, equipped with extending acoustic probes, and where the said devices interact with 3-D array of ultrasonic transducers and with 2-D inspection portable ultrasound detecting devices by means of Data Processing System.

6. Method as defined in claims 1, 3, 4 and 5 wherein all the said ultrasound inspection techniques are being applied with priority of portable and hand-held measures either inside of overcrowded enclosed creatures' inhabiting areas, featured with interfering background noises, or on the outside, featured with availability of chaotic and mixing movement inside a total crowd of creatures. The data interaction among portable and hand-held measures is being kept continuously, where the use of Local 1-D Pinpointing Technique is being predicted by running results of Portable 2-D Inspection Technique application.

7. Method as defined in claims 1-6 wherein operating interrelation among all the stationary surveying, portable inspection and local pinpointing procedures are being fulfilled according the following schedule:
a) The preliminary choosing of ultrasonic devices for all the operational techniques is being accomplished regarding compliance of their specification figures (at least: directivity, sensitivity, selectivity, remote ability, and S/N ratio) to the operational conditions of acquiring ultrasound signs of respiratory diseases in fowls, mammals and another breathing creatures.
b) Stationary 3-D Surveying technique is being in action all the time and it interacts continuously with Portable 2-D Inspection technique. The direction, where the sampled ultrasound emission comes from, is being defined as a direction from nearest operator to the suspected sector, hence the said operator and sector happened to be adjacent at the moment of the said signal appearance.
c) Portable 2-D Inspection technique is being in action continuously. The short-distance inspection is being fulfilled by at least three operators, which follow the routes, indicated previously to pass around every sector of an entire creatures' inhabiting area. The double-way interaction among 3-D and 2-D techniques, and Data Processing System is being maintained continuously. Since 3-D technique detects direction of emission of a typical ultrasound sign of respiratory disease, Data Processing System aims at least one of the operators to inspect a sick suspected sector, while other operators continue their routine walk the rounds.
d) Whether short-distance 2-D technique proves the presence of respiratory illness in a sick suspected sector, the involved operator starts to apply Local 1-D Pinpointing Technique. In the case that the sick suspected unit group is trustworthily pinpointed, this involved operator stays thereat till sick creatures have been withdrawn. Should the number of vectored by 3-D technique the suspected directions will exceed the number of operators, all the competing ultrasound sources are to be stopped for repeated verification of suspected directions. Anyway, at that extreme case the priority of application of 2-D and 1-D techniques ought to be given to infected places, which are located closer to the central part of an overcrowded creatures' inhabiting section of an entire area, preferably to the central part of the suspected sector, adjacent to an operator at the very moment.
e) During application of Local 1-D Pinpointing Technique in one of the sectors another 3-D and 2-D techniques continue to function in accordance with scheduled routine.
